(19) United States
(12) Patent Application Publication
(10) Pub. No.: US 2004/0043442 A1
(43) Pub. Date: Mar. 4, 2004

(54) Use of betaine in functional products having blood pressure lowering effects

(75) Inventors: Kirsti Jutila, Espoo (FI); Matti Uusitupa, Kuopio (FI); Ursula Schwab, Kuopio (FI)

Correspondence Address: SCULLY SCOTT MURPHY & PRESSER, PC, 400 GARDEN CITY PLAZA, GARDEN CITY, NY, 11530

(73) Assignee: Finnfeeds Finland Oy, Espoo (FI)

(21) Appl. No.: 10/617,974

(22) Filed: Jul. 11, 2003

Related U.S. Application Data
(63) Continuation of application No. PCT/FI02/00024, filed on Jan. 11, 2002.

(30) Foreign Application Priority Data
Jan. 12, 2001 (FI) 20010075

(51) Int. Cl. C12Q 1/34

(52) U.S. Cl. 435/18

(57) ABSTRACT

The present invention relates to the use of betaine in functional products, such as pharmaceutical products, functional food products, food supplements, natural products and the like. In particular, the present invention relates to the use of betaine for the manufacture of functional products having blood pressure lowering effects, and to methods for lowering blood pressure.

This application is a Continuation of International Application PCT/FI02/00024 filed on Nov. 1, 2002, which designated the U.S. and was published under PCT Article 21(2) in English.

### FIELD OF THE INVENTION

The present invention relates to the use of betaine in functional products, such as pharmaceutical products, functional food products, food supplements, natural products and the like. In particular, the present invention relates to the use of betaine in functional products having blood pressure lowering effects, and to methods for lowering blood pressure.

### BACKGROUND OF THE INVENTION

Betaine has been reported to both improve gut health and to increase the food intake and growth of animals. Betaine has also been found to decrease the body fat of for example fish, chicks, piglets and growing pigs (see e.g. Virtanen, E. et al., Effects of food containing betaine/amino acid additive on the osmotic adaption of young Atlantic Salmon, Salmo salar L. Aquaculture 83 (1989) 109-112; Saundersson, C. L. and McKinlay, J., Changes in body weight, composition and hepatic enzyme activities in response to dietary methionine, betaine and choline levels in growing chicks, British J. Nutriton 63 (1990) 339-349; and Virtanen, E. and Campbell, R., Reduzierung der Ruckenspeckdicke durch Einsatz von Betain bei Mastschweinen (Reduction of backfat thickness through betaine supplemenetation of diets for fattening pigs). Handbuch der tierischer Veredlung. Verlag H. Kamlage, Osnabruck, Deutschland, 19 (1994) 145-150).

Betaine has also been reported to have pharmacological effects in animals. For example proline betaine has been reported to prevent perosis in chicks and glycine betaine has been reported to prevent the detrimental effects of coccidiosis in broilers (PCT/FI94/00166). Betaine has also been reported to enhance the reproductive performance of animals (PCT/FI96/00211).

In man, betaine has been reported to reduce increased blood homocysteine levels in patients suffering from any of the three primary types of homocysteinuria (see e.g. Wilcken, D. E. L., Dudman, N. P. B. and Tyrrell, P. A., Homocystinuria Due to Cystathionine β-Synthase Deficiency—The Effects of Betaine Treatment in Pyridoxine-Responsive Patients. Metabolism 34 (1985) 12:1115-1121; Surtees, R., Bowron, A., Leonard, J. Pediatr Res. 42 (1997) 577-582). The effect of betaine on plasma homocysteine levels in healthy subjects has also been studied (Brouwer, I. A. and Urgert, B. Betaine Supplementation on Plasma Homocysteine in Healthy Volunteers. Arch. Intern Med 160 (2000) 2546-2547). The positive effect of betaine on the methionine level and transamination thereof has been reported by A. Tangerman et al. (Tangerman, A., Wilcken, B, Levy, H. L., Boers, G. H. J., and Mudd, S. H. Methionine Transamination in Patients With Homocystinuria Due to Cystathionine β-Syntase Deficiency. Metabolism 49 (2000) 8:1071-1077).

A betaine preparation for the treatment of homocystinuria is commercially available under the trademark Cystadane, Orphan Medical Inc., Minnetonka, Minn. 55305. The product consists of anhydrous betaine as a white, granular, hygroscopic powder, which is easily soluble in water. For use, a prescribed amount of the powder is added to water, mixed until completely dissolved, and then immediately orally consumed (Orphan Medical Inc., 13911 Ridgedale Drive, Suite 475, Minnetonka, Minn. 55305, Cyst 06, revised 1196). Other pharmaceutical preparations for reducing homocysteine levels, and optionally containing betaine as one of the ingredients, are described e.g. in WO 00/44385, Bogye Gabor, and EP 0 595 005 A1, Vesta Medicines Ltd.

A pharmaceutical preparation for the treatment and prevention of transmethylation disorders, such as cardiovascular diseases, is described in WO 00/25764, Merck G.m.b.H. The composition preferably comprises three different active components, namely a methyl or methylene donor, a methyl transporter, and a bioflavonoid. As an example, a composition containing betaine 600 mg, Ca L-5-methyltetrahydrofolate 0.5 mg and isoquercetin 500 mg, is described.

Betaine has also been reported to be useful in treating hepatopathies, in particular fatty liver (Babucke, G. and Sarre, H. Klinische Erfahrungen mit Betaincitrat. Med. Klin. 68 (1973) 1109-1113). Betaine has also been suggested to decrease the concentration of serum triglycerides and blood alcohol. However, there are no controlled studies regarding these issues.

RU 2105509, Dal'nevostochnyj kommercheskij institut, describes a beetroot juice called “Red Beet Rose”. The juice contains sugar syrup, citric acid, and juice from the top parts of beetroots. For preparation of the juice, top parts of beetroot are washed, treated with steam in 105 C, ground and pressed. The pressed juice is filtered and mixed with sugar syrup and citric acid. The advantage of the described invention is that the top parts of beetroots, which according to the publication earlier have been regarded as waste material, now can be utilized and thus the assortment of vegetable juices be broadened, and a product with beautiful cherry colour obtained.

High blood pressure, or hypertension, is one of the most common diseases in developed countries. High blood pressure is defined as a consistent recording of systolic blood pressure of 140 mmHg or higher, diastolic blood pressure of 90 mmHg or higher. The two main factors influencing blood pressure are the peripheral resistance, i.e. the width of the arteries into which the blood is being pumped, and the cardiac minute volume, i.e. the amount of blood the hearth pumps. However, blood pressure is very complicatedly regulated and many other factors also play an important role.

Most people having high blood pressure have primary or essential hypertension where the ethiology is unknown and lifestyle factors play a significant role. Only a small number of cases of high blood pressure are caused by other illnesses, such as kidney disease or hormonal imbalance (secondary hypertension).

Clinical trials have shown that reductions in elevated blood pressure of about 10-12 mmHg systolic and 5-6 mmHg diastolic conferred relative reductions in stroke risk of 38% and in risk of coronary heart disease of 16% (Collins R., MacMahon S. Blood pressure, antihypertensive drug treatment and the risks of stroke and of coronary heart disease. Br Med Bull 50 (1994) 272-98). Hypertensive patients are commonly treated with diuretics, β-blockers, calcium antagonists and ACE (angiotensin converting enzyme) inhibitors. The treatment is chosen individually and is always kept under doctor's control. As an alternative to medication, or in addition thereto, other methods to lower elevated blood pressure levels have been investigated, and nowadays the main aim is to prevent that the blood pressure raises to a level where medication is needed. Lifestyle factors and daily diet play an important role in the pathogenesis of essential hypertension. Thus, the blood pressure lowering effects of dietary components are under intensive investigation. The use of functional foods as a part of the normal diet, for maintaining a normal blood pressure, would be an appreciated alternative for the consumers.

In the literature of the art it is traditionally known that peptide like products may reduce blood pressure. E.g. taurine is known to lower blood pressure in essential hypertension and some experimental hypertensive models (Harada, H., Kitazaki, K., Tsujino, T., Watari, Y., Iwata, S., Nonaka, H., Hayashi, T., Takeshita, T., Morimoto, K., Yokoyama, M. Oral taurine supplementation prevents the development of ethanol-induced hypertension in rats. Hypertens Res 23 (2000) 3:277-284). Arginine also seems to lower blood pressure and may inhibit atherogenesis. However, the evidence of the beneficial effects of these two compounds in human are scarce (Niittynen, L., Nurminen, M. L., Korpela, R., Vapaatalo, H., Role of arginine, taurine and homocysteine in cardiovascular diseases. Ann Med 31 (1999) 5:318-326).

Thus, there is still a constant and obvious need to provide new functional products, which have a blood pressure lowering effect. Ideally, said products should be suitable both for maintaining normal blood pressure and for treating hypertension. Furthermore, the products should be of a type familiar to the consumer and easily used as a part of the normal diet.

### SHORT DESCRIPTION OF THE INVENTION

Consequently, the object of the present invention is to provide a product having such properties. According to the present invention, this object is achieved by the use of betaine.

The present invention thus relates to the use of betaine for the manufacture of a product for maintaining normal blood pressure or for reducing (elevated) blood pressure.

Especially, the present invention relates to the use of betaine for the manufacture of a product for the treatment or prevention of hypertension.

In a preferred embodiment of the invention, the product is an edible product, such as a pharmaceutical product, a food product, a food supplement, a dietary supplement, or a natural product.

The present invention also relates to a method for maintaining normal blood pressure, comprising administering to a subject betaine in an amount sufficient to achieve the desired result.

Furthermore, the present invention relates to a method for reducing (elevated) blood pressure, comprising administering to a subject betaine in an amount sufficient to achieve the desired result.

The present invention also relates to a method for the prevention or treatment of hypertension, comprising administering to a subject in need of such treatment betaine in an amount sufficient to achieve the desired result.

In a preferred embodiment of the present invention, glycine betaine in the form of betaine anhydride, betaine monohydrate, or a betaine salt, is used. In another preferred embodiment, the betaine used in accordance with the present invention is prepared synthetically or by chromatographic separation, and, if necessary or desired, converted into a salt or derivative.

In accordance with the present invention, it has been shown that betaine lowers the diastolic blood pressure, in particular.

### DETAILED DESCRIPTION OF THE INVENTION

The present invention is based on the finding that betaine has a very beneficial lowering effect on blood pressure, and in particular on the diastolic pressure.

Betaine refers to fully N-methylated amino acids. Betaines are natural products that have an important function in both plant and animal metabolism. One of the most abundant betaines is a glycine derivative in which three methyl groups are attached to the nitrogen atom of the glycine molecule. This betaine compound is usually called betaine, glycinebetaine, trimethylglycine or trimethyl-ammonium acetate, and it has the following structural formula:

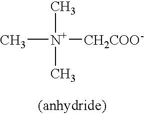

Other betaines include, for example, alanine betaine, proline betaine and histidine betaine. A detailed description of betaines is given by Wyn Jones, R. G. and Storey, R. in The physiology and Biochemistry of Drought Resistance in Plants, ed. Paleg, L. G. and Aspinall, D. Academic Press, Sydney, Australia, 1981, which is incorporated herein by reference.

Betaine thus has a bipolar structure and it contains several chemically reactive methyl groups, which it can donate in enzyme-catalyzed reactions. Most organisms are able to synthesize small quantities of betaine e.g. for the methyl function, but they are not able to produce and store large quantities of betaine. The best-known organisms that accumulate betaine are plants of the genus Chenopodiaceae, such as sugar beet, and some microbes and marine invertebrates. Probably the main reason for these organisms to store betaine is that betaine functions as an osmolyte and thereby protects the cells from the effects of osmotic stress. Betaine has also been observed to stabilize the operation of macromolecules in cell membranes.

Human cells also contain betaine. In the human body, betaine likewise is involved in methionine metabolism: it donates a methyl group to homocysteine, which in turn turns to methionine. The amount of betaine in different organs vary a lot, high amounts are present e.g. in Kupfer cells in the liver and in kidney cells, and betaine is present both in blood and in urine. In plasma from healthy humans, the betaine concentration is about 20-60 umol/l, the concentration being considerable higher in adult males than in adult females. In urine, the betaine concentration is significantly high in neonatals and children under 12 months old, the concentration decreasing, after an initial sharp decline, steadily during childhood (Lever, M., Sizeland, P. C. B., Bason, L. M., Hayman, C. M. and Chambers, S. T. Glycine betaine and proline betaine in human blood and urine. Biochimica et Biophysica Acta 1200 (1994) 259-264).

Betaine is thus a compound naturally occurring in human cells, and it is part of our normal diet, present in foods such as sugar beet, spinach, and seafood, in particular molluscs and crustacean.

Betaine has been safely used in animal feed for over 25 years. Betaine was first used in fish feeds, for salmon and trout, as an attractant and an osmoprotectant during the freshwater/seawater transfer stage (Virtanen et al., 1989, supra). Its use has developed in the last 10 years as an additive for poultry and pig feed as a methyl donor and osmoprotectant. Some studies have indicated that betaine is able to decrease the amount of fat tissue in pigs without affecting the amount of lean tissue (Virtanen and Campbell, 1994, supra).

Betaine also has been used for several years in the treatment of homocystinuria, and has been demonstrated to be safe, also for use in pediatrics, and without severe adverse effects.

Betaine can be obtained, for example, from sugar beet by chromatographic methods (see e.g. WO 97/45185, Cultor Ltd., and the description of the background art given therein, particularly on p. 4, I. 9-26; EP 54544 Suomen Sokeri Oy; EP 345511, Suomen Sokeri Oy). Alternatively, betaine can be prepared synthetically, by using organic synthesis, biosynthesis or genetechnology. Suitable synthesis routes are described e.g. in WO 00/11142, Cultor Corporation. Betaine is commercially available from Finnfeeds Finland Ltd. Betaine products, such as anhydrous, monohydrate, hydrochloride and concentrated betaine solutions, are also available and can be used in the way described in the current document. Other betaine salts and derivatives can also be used. As examples may be mentioned, in a non-exclusive manner, betaine glutamate or betaine citrate.

Preferably, the betaine product is prepared by chromatographic methods, and, when desired, converted into a salt or derivative. Sugar beet contains about 0.20% glycine betaine and is regarded as the most preferred starting material for betaine production.

For use in accordance with the present invention, betaine or a salt thereof is administered to the subject as such or as a component of a pharmaceutical product, a food product, a food supplement, a dietary supplement or a natural product. Preferred administration forms include, but are not limited to, pills, capsules, dry powders, granulates, and liquid formulas. Betaine is readily dissolved in water or beverages of any kind, such as fruit juices, vegetable juices, nectars, milk products, soft drinks, coffee or tea, beer and other alcoholic drinks, etc. It can also be used in dry form e.g. in milk powder, or instant coffee, tea or cocoa. Another major product group comprises salt and sweet snacks of any kind, peanuts, energy bars, hard candy, liquorice sticks and powders, ammonium chloride in liquid, powder or pastille form, confectionery, cookies, chewing gums, dried products, such as dried fruits, vegetables, raisins, and the like. For use in the preparation of pharmaceutical products, food products, dietary additives, and natural products, betaine in the form of dried powder, granulate or (water) solution is considered as preferred; in these forms, it is easily added to the final product either during or after the preparation thereof.

In connection with the present invention, the term food is broadly construed, including any edible products which can be in a liquid to solid form, and covering both ready-to-eat products and products to which the product of the invention is added in connection with consumption, as a supplement or to be a constituent of the product. For instance, foods can be products of beverage industry, confectionery industry, food processing industry, meat-processing industry, fish processing industry, baking industry, and dairy industry. As mentioned above, beverages and snack products are regarded as preferred.

The final products comprising betaine as a blood pressure lowering substance in accordance with the present invention may thus include, in addition to the betaine, ingredients normally used in such products, and they are prepared according to methods conventionally used in pharmaceutical industry, and food and dietary supplements industry, including beverages and functional products.

Betaine is used in an amount sufficient to achieve the desired, blood pressure maintaining or lowering effect. The amount can vary within a large range, depending e.g. on the health, age, and medication of the subject, and can easily be determined by the physician after the publication of the invention. Suitable added amounts may be e.g. about 0.05-20 g betaine per day. Betaine amounts of 0.1-8 g per day, in particular 0.5-6 g per day, are regarded as preferred.

In accordance with the present invention, it has been shown that betaine lowers the blood pressure. The examples show that a significant reduction in blood pressure is achieved. Furthermore, the diastolic pressure, in particular, is lowered. This is an important finding, bearing in mind that most pharmaceutical products presently on the market mainly lower the systolic blood pressure.

It should also be noted that betaine exerts its blood pressure lowering effect even in normotensic subjects, i.e. people with normal blood pressure. The blood pressure lowering effect of betaine is expected to be even more significant in hypertensive subjects. It is also believed that betaine has favourable effects for instance on the oral mucosa, in prevention of cardiovascular disease, as liver protectant, and as osmoprotectant in the kidneys. Betaine is thus suitable for use both as a pharmaceutical agent and as a functional agent having beneficial effects on our overall well-being.

Significantly increased amounts of betaine were found in the blood and urine of the persons given betaine in the study. This finding indicates that betaine is absorbed and secreted trough the kidneys. There was, however, no clear correlation between blood and urine betaine levels. After administration of 1, 3 or 6 g betaine an amount of about 3, 5 and 7%, respectively, was recovered as betaine and dimethylglycine (DMG) in 24 h urine. After loading betaine 100 mg/kg, peak serum concentrations were found 1-2 h after loading; the concentrations varying a lot with indiviudual (148-258 μmol/l).

The invention will be described in greater detail by means of the following examples. The examples are only provided in order to illustrate the invention and they should not be construed to restrict the scope of invention in any way.

### EXAMPLE

The effects of betaine supplementation on blood pressure, and in addition on body weight, body composition, resting energy expenditure, and concentrations of serum total and lipoprotein lipids and plasma homocysteine in humans, were studied. The participants were healthy, obese adults with normal blood pressure.

Forty-two subjects (14 males, 28 females) without any chronic disease and with normal liver, kidney and thyroid function participated in the study. The inclusion criteria for body mass index (BMI) was 28-40 kg/m² and for age 25-60 years. Serum concentration of total triglycerides had to be <3.5 mmol/l and that of total cholesterol <7.5 mmol/l. Inclusion criteria for fasting concentration of plasma glucose was <6.7 mmol/l. Subjects with lipid lowering medication were excluded as well women with perimenopause. Four women were postmenopausal in the control group and 6 were postmenopausal in the betaine group. None of the participants were on a medication for hypertension.

The subjects were not allowed to use nutrient supplements during the study or one month before the study.

Study Design

The study was a controlled, randomized double-blinded parallel study. Before the 12-week intervention period the subjects had a 4-week run-in period during which they consumed 1 dl/d orange juice with 6 g grapefruit juice per 1 dl orange juice twice a day (1 dl in the morning, 1 dl in the evening). Grapefruit juice was added to simulate the bitter taste of betaine. The subjects were on their regular eucaloric diet during this period. For the intervention period with a hypocaloric (−2100 kJ or −500 kcal) diet the subjects were randomly assigned in two groups (20 for the control group, 22 for the betaine group). The subjects were matched for BMI, gender and menstrual cycle.

The control group consumed orange juice 1 dl twice a day and the betaine group consumed betaine enriched orange juice 1 dl twice a day. The amount of betaine was 3 g per 1 dl orange juice, giving a daily betaine dose of 6 g.

Statistical Analyses

The data were analyzed with the SPSS/PC+ statistics program (V8.0, SPSS; Chicago, Ill.). Before further analyses, normal distribution of the variables was checked with the Kolmogorov-Smimov test with Lilliefors significance correction. Variables with abnormal distribution (TG) were logarithmized and the logarithmized values were used in further analyses. GLM for repeated measures was used to test the changes within time. In cases the result of this analysis was significant paired t-test was used for two-tailed comparisons within the groups. Student's t-test was used for between-group comparisons. Mann-Whitney's U-test and Wilcoxon matched-pairs signed-ranks test, respectively, were used to test the changes in lean body mass as kg, lean body mass as percent and fat mass as percent measured by bioelectric impedance since the distribution of these variables did not turn to normal by logarithmization or other arithmetic procedures. All data are expressed as mean±SD.

Effect of Betaine on Blood Pressure

Blood pressure was measured from the right arm after five minutes of rest in sitting position using a zero mercury sphygmomanometer. Two measurements were performed and the mean of them was calculated and used for further analyses.

The results are presented in tables 1 and 2. The blood pressure, especially the diastolic blood pressure decreased significantly in the betaine group (Table 1). The results thus clearly show the effect of betaine on diastolic blood pressure reduction even in normotensive subjects; it is expected that the effect is even more significant in (mildly) hypertensive subjects.

TABLE 1
Blood pr ssure at the beginning (4 wk) and at the nd (16 wk)
of the int rvention period

| Blood pressure (mmHg) | 4 wk | 16 wk | P-value |
|---|---|---|---|
| | Control group | | |
| Systolic | 127.4 ± 17.5 | 126.8 ± 18.1 | 0.693 |
| Diastolic | 86.1 ± 10.8 | 83.7 ± 11.7 | 0.110 |
| | Betaine group | | |
| Systolic | 122.5 ± 9.5 | 121.1 ± 9.4 | 0.432 |
| Diastolic | 85.4 ± 7.9 | 80.5 ± 7.1 | 0.002 |

Mean ± SD.

TABLE 2
Change in blood pressure during the intervention period.

| | Control group | Betaine group | P-value |
|---|---|---|---|
| Blood pressure (mmHg) | | | |
| Systolic | −0.6 ± 6.7 | −1.4 ± 8.0 | 0.740 |
| Diastolic | −2.4 ± 6.4 | −4.9 ± 6.6 | 0.219 |

Mean ± SD.

Body weight was measured using the same calibrated electronic scale throughout the study. Body composition was measured by a bioelectric impedance (BIA 101S with Bodygram software, Akern S.r.I. Biosearch, Italy). The results are presented in table 3.

When changes in the above mentioned variables during the intervention period were compared between the groups no significant differences were found (table 4). The results thus seem to be due to the lower calory intake, and not to betaine intake. This result confirms that the blood pressure lowering effect stems from betaine intake, and not from an overall weight reduction effect.

TABLE 3
Body weight, and BMI at the beginning (4 wk) and at the end
(16 wk) of the intervention period

| | 4 wk | 16 wk | P-value |
|---|---|---|---|
| | Control group | | |
| Body weight (kg) | 94.6 ± 9.9 | 91.1 ± 8.7 | 0.001 |
| BMI (kg/m²) | 33.2 ± 3.2 | 32.1 ± 3.0 | 0.001 |
| | Betaine group | | |
| Body weight (kg) | 95.7 ± 11.3 | 93.5 ± 11.1 | 0.004 |
| BMI (kg/m²) | 33.5 ± 3.2 | 32.8 ± 3.7 | 0.005 |

TABLE 4
Change in body weight and BMI in the intervention period.

| | Control group | Betaine group | P-value |
|---|---|---|---|
| Body weight (kg) | −3.4 ± 4.1 | −2.2 ± 3.2 | 0.303 |
| BMI (kg/m²) | −1.2 ± 1.3 | −0.7 ± 1.1 | 0.230 |

Plasma total homocysteine was determined by a modification of the highpressure liquid chromatographic method described by Ubbink et al. (Ubbink, J. B., Vermaak, W. J. H., Bissbort, S. Rapid high-performance liquid chromatographic assay for total homocysteine levels in human serum. J Chromatogr (1991) 565:441446). The modified mobile phase consisted of 0.37 mol/l acetate and 0.5% methanol, pH 4.15.

Folate concentration of plasma and erythrocytes was determined by the fluorescence polarization immunometric Imx-method (Abbott Laboratories, IL).

Serum normal homocysteine concentration (<15 μmol/l) decreased significantly in the betaine group (8.76±1.63 vs. 7.93±1.52, 4 wk vs. 16 wk, P=0.025). In the control group it did not change ((8.01±2.47 vs. 8.12.±2.25, P=N.S.). The change in serum homocysteine concentration during the intervention period was also significantly different between groups (0.17±0.27 vs. −0.83±0.34, P=0.030). Fasting plasma folate concentrations did not change during the study in either of the groups whereas the folate concentration in erythrocytes decreased significantly (P=0.024) in the betaine group but did not change in the control group. Thus, the serum or erythrocyte folate levels of the subjects did not cause the homocysteine lowering effect. Consequently, the advantageous effect of betaine alone on the homocysteine level has been clearly proved in connection with the present invention.

The study also showed that the 6 g daily dose was well tolerated and no side effects were observed.

1. Method for maintaining normal blood pressure, comprising administering to a subject glycine betaine in an amount sufficient to achieve the desired result.

2. The method according to claim 1, wherein a daily dosage of 0.05-20 g of glycine betaine is administered.

3. The method according to claim 1, wherein a daily dosage of 0.1-8 g per day of added glycine is administered.

4. The method according to claim 1, wherein glycine betaine is in the form of anhydride, monohydrate, or a salt.

5. Method for reducing blood pressure, comprising administering to a subject glycine betaine in an amount sufficient to achieve the desired result.

6. The method according to claim 5, wherein a daily dosage of 0.05-20 g of glycine betaine is administered.

7. The method according to claim 5, wherein a daily dosage of 0.1-8 g per day of added glycine is administered.

8. The method according to claim 5, wherein glycine betaine is in the form of anhydride, monohydrate, or a salt.

9. The method according to claim 5, wherein the reduction of diastolic pressure is achieved.

10. Method for the prevention or treatment of hypertension, comprising administering to a subject in need of such treatment glycine betaine in an amount sufficient to achieve the desired result.

11. The method according to claim 10, wherein a daily dosage of 0.05-20 g of glycine betaine is administered.

12. The method according to claim 10, wherein a daily dosage of 0.1-8 g per day of added glycine is administered.

13. The method according to claim 10, wherein glycine betaine is in the form of anhydride, monohydrate, or a salt.

14. The method according to claim 10, wherein a daily dosage of 0.05-20 g of glycine betaine is provided.

15. The method according to claim 10, wherein a daily dosage of 0.1-8 g per day of added glycine is provided.

16. The method according to claim 10, wherein the added glycine betaine is in the form of anhydride, monohydrate, or a salt.

17. The method according to claim 10, wherein the reduction of diastolic pressure is achieved.
